(19) United States
(12) Patent Application Publication
(10) Pub. No.: US 2021/0101017 A1
(43) Pub. Date: Apr. 8, 2021

(54) WEARABLE DEVICE FOR NON-INVASIVE THERAPY OF MAMMARY GLANDS

(71) Applicant: LIGHT TREE, Amstelveen (NL)

(72) Inventors: ALAIN DIJKSTRA, Amstelveen (NL); YVONNE JOHANNA MARGARETHA HOUTHUIJS, Amstelveen (NL)

(73) Assignee: LIGHT TREE, Amstelveen (NL)

(21) Appl. No.: 16/926,653

(22) Filed: Jul. 11, 2020

(30) Foreign Application Priority Data
Oct. 2, 2019 (IN) 201911039861

(51) Int. Cl. A61N 5/06; A41C 3/00; A41C 3/04

(52) U.S. Cl. CPC A61N 5/0613; A41C 3/0064; A41C 3/04; A61B 5/4312; A61N 2005/0652; A61N 2005/0626; A61N 2005/0653; A61N 2005/0645

(57) ABSTRACT

A wearable device for mammary glands in human beings, comprises a first brassiere cup and a second brassiere cup, the first brassiere cup including a first inner surface and the second brassiere cup including a second inner surface, a first layer of a diaphanous material attached to the first inner surface and a second layer of the diaphanous material attached to the second inner surface, a plurality of first openings provided in the first layer and a plurality of second opening provided in the second layer, a plurality of first Light Emitting Diodes (LEDs) provided between the first inner surface and the first layer, a plurality of second LEDs provided between the second inner surface and the second layer, a sensor unit adapted to determine one or more physiological parameters and an underwire connecting the first and the second brassiere cups.

### TECHNICAL FIELD

The present invention relates generally to wearable therapeutic devices. More specifically, the present invention relates to wearable devices for supporting mammary glands of an adult human female, wherein brassiere cups of such wearable devices are enabled with low power light stimulation for treating physiological conditions including tumors of the mammary glands.

### BACKGROUND ART

Mammary glands of an adult human female are tissues that overlie pectoral muscles in the chest region on the upper torso. Particularly, the mammary glands of an adult human female are made of specialized tissues. These specialized tissues are primarily made of fat, and their primary function is to facilitate the production of milk thereby becoming glandular tissues. The characteristic shape of the mammary glands is due to the connective tissues and ligaments that connect them with the body. Being glandular tissues, the mammary glands are prone to several diseases and disorders including but not limited to cysts, infections, and inflammations. The types of cysts related conditions that may manifest within the mammary glands include chronic cystic mastitis, diffuse cystic mastopathy, and mammary dysplasia, among others.

The infections and inflammations of the mammary glands may be caused due to several factors including trauma, secretory stasis or milk engorgement, hormonal stimulation, infections, or autoimmune reactions. The infections of the mammary glands also include bacterial mastitis, chronic subareolar abscess, tuberculosis of the mammary glands, and syphilis of the mammary glands, retro-mammary abscesses, actinomycosis of the mammary glands, duct ectasia syndrome, and mammary gland engorgement. Other mammary gland conditions include Mordor's disease, Paget's disease of the breast, nipple discharge, galactorrhea, mastalgia, and galactocoele, to name a few. Also, many women face the problem of nipple fissures and inflammation of mammary glands during the lactation time in the postpartum period.

The tumors appearing in the mammary glands can be either benign tumors or malignant tumors. The benign tumors exhibit a relatively slow growth rate and do not spread into the surrounding areas of the affected portion of the mammary glands. On the other hand, malignant tumors exhibit rapid growth and can extend to other organs and tissues surrounding the affected area.

Many kinds of therapies are known in the prior art literature for treating mammary gland abnormalities and disorders including the tumors. One of the solutions is a surgical method comprising reconstruction of the affected mammary gland with implants made of synthetic materials like silicone. Silicone devices filled with silicone gel or saline are used to reshape the affected mammary gland. However, a plurality of risk factors follows the implanted mammary gland. These risk factors include among others, asymmetry of the implanted mammary gland with the natural mammary gland, pain in the implanted area, bleeding, infection in the implanted area, and loss of sensation, to name a few. As another alternative solution, radiation therapy is also used.

However, there is a risk of damage to the tissues of surrounding organs like lungs and heart. Further, radiation therapy is not capable of killing all cells in the tumor. Still, further, there is an increased risk of wound complications and poor healing. As another alternative solution, chemotherapy is used for the treatment of the tumors, wherein a suitable drug is administered to the affected area. As each drug has its own set of side effects which differs and varies from person to person, chemotherapy is shown to exhibit a plurality of side effects. These side effects include nausea, bleeding, hair loss, and constipation, to name a few.

Therefore, given the foregoing disadvantages inherent in the known disclosures of the prior art and in response to the shortcomings of the prior art, there is long felt a need for a wearable device for the light-based treatment of disorders and diseases associated with the mammary gland of an adult human female and which has little or no side effects and which further provides a completely a pain-free, non-invasive, and simple to use therapy for treating the conditions of the mammary gland of an adult human female.

### OBJECTS OF THE INVENTION

Some of the objects of the present invention are as stated below:

An object of the present invention is to provide a wearable device for the treatment of disorders and diseases associated with mammary glands of an adult human female wherein the treatment is light-based;

Another object of the present invention is to provide a wearable device for the treatment of disorders and diseases associated with the mammary gland wherein such therapy has little or no side effects;

Yet another object of the present invention is to provide a wearable device for the treatment of disorders and diseases associated with the mammary gland, wherein such therapy is a pain-free therapy;

A further object of the present invention is to provide a wearable device for the treatment of disorders and diseases associated with the mammary gland, wherein such therapy is a non-invasive therapy; and

An additional object of the present invention is to provide a wearable device for the treatment of disorders and diseases associated with the mammary gland, wherein the wearable device is simple to use.

Other objects, aspects, features, and goals of the present invention will be better understood from the following detailed description.

### SUMMARY

According to a first aspect of the present invention, there is provided a wearable device for mammary glands in human beings, the wearable device comprising a first brassiere cup and a second brassiere cup, the first brassiere cup including a first inner surface and the second brassiere cup including a second inner surface, a first layer of a diaphanous material attached to the first inner surface and a second layer of the diaphanous material attached to the second inner surface, wherein the first and the second layers are adapted to be in contact with the mammary glands, a plurality of first openings provided in the first layer and a plurality of second opening provided in the second layer, a plurality of first Light Emitting Diodes (LEDs) provided between the first inner surface and the first layer, the plurality of first LEDs provided within the plurality of respective first openings, a plurality of second LEDs provided between the second inner surface and the second layer, the plurality of second LEDs provided within the plurality of respective second openings, a sensor unit adapted to determine one or more physiological parameters and an underwire connecting the first and the second brassiere cups.

In one embodiment of the invention, the plurality of first LEDs and the plurality of second LEDs are configured to emit electromagnetic radiation in one or more of a pulse mode and continuous mode.

In one embodiment of the invention, the plurality of first LEDs and the plurality of second LEDs have been provided on an Organic LED (OLED) based flexible panel or an inorganic LED based flexible panel.

In one embodiment of the invention, the plurality of first LEDs and the plurality of second LEDs are provided as a printable composition of micro-LEDs, printed on a substrate.

In one embodiment of the invention, the wearable device further comprises a processor, a memory unit and a communication unit, the memory unit including machine-readable instructions that when executed by the processor, enable the processor to receive physiological data from the sensor unit and regulate emission characteristics of the plurality of first LEDs and the plurality of second LEDs in correlation with the physiological data.

In one embodiment of the invention, the processor is further enabled to transmit the physiological data to an external electronic communication device, via the communication unit.

In one embodiment of the invention, the processor is further enabled to receive a control input, and regulate emission characteristics of the plurality of first LEDs and the plurality of second LEDs in correlation with the physiological data and the control input.

According to a second aspect of the present invention, there is provided a method of utilizing a wearable device for mammary glands in human beings, the wearable device comprising a first brassiere cup and a second brassiere cup, the first brassiere cup including a first inner surface and the second brassiere cup including a second inner surface, a first layer of a diaphanous material attached to the first inner surface and a second layer of the diaphanous material attached to the second inner surface, wherein the first and the second layers are adapted to be in contact with the mammary glands, a plurality of first openings provided in the first layer and a plurality of second opening provided in the second layer, a plurality of first Light Emitting Diodes (LEDs) provided between the first inner surface and the first layer, the plurality of first LEDs provided within the plurality of respective first openings, a plurality of second LEDs provided between the second inner surface and the second layer, the plurality of second LEDs provided within the plurality of respective second openings, a sensor unit adapted to determine one or more physiological parameters, and an underwire connecting the first and the second brassiere cups, the method comprising steps of administering a photosensitizing drug to one or both of the mammary glands, receiving physiological data from the sensor unit and regulating emission characteristics of the plurality of first LEDs and the plurality of second LEDs in correlation with the physiological data.

In one embodiment of the invention, the method further comprises a step of transmitting the physiological data to an external electronic communication device.

In one embodiment of the invention, the method further comprises a step of receiving a control input and regulating emission characteristics of the plurality of first LEDs and the plurality of second LEDs in correlation with the physiological data and the control input.

In the context of the specification, the term “diaphanous materials” refers to the materials that allow the transmission of electromagnetic radiation, including at least Ultra-Violet (UV), visible light, and Infrared (IR), through them.

### BRIEF DESCRIPTION OF ACCOMPANYING DRAWINGS

So that the manner in which the above-recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein::

FIG. 1 illustrates an anatomical sectional view of a mammary gland of an adult human female;

FIG. 2 illustrates an exploded view of a wearable device for mammary glands, in accordance with an embodiment of the present invention;

FIG. 3A illustrates a front view of the wearable device of FIG. 2;

FIG. 3B illustrates a partial side sectional view of the wearable device of FIG. 2;

FIG. 4 illustrates a control architecture for monitoring and control of the wearable device of FIG. 2, in accordance with an embodiment of the present invention; and

FIG. 5 illustrates a method for utilizing the wearable device of FIG. 2, in accordance with an embodiment of the present invention.

### DETAILED DESCRIPTION

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claims. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned.

Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents acts, materials, devices, articles, and the like is included in the specification solely to provide a context for the present invention. It is not suggested or represented that any or all of these matters form part of the prior art base or were common general knowledge in the field relevant to the present invention. In this disclosure, whenever a composition or an element or a group of elements is preceded with the transitional phrase “comprising”, it is understood that we also contemplate the same composition, element or group of elements with transitional phrases “consisting of, “consisting”, “selected from the group of consisting of, “including”, or “is” preceding the recitation of the composition, element or group of elements and vice versa.

The present invention is described hereinafter by various embodiments with reference to the accompanying drawings, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only and are not intended to limit the scope of the claims. Also, several materials are identified as suitable for various facets of the implementations. These materials are to be treated as exemplary and are not intended to limit the scope of the invention.

It is to be noted here that in the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelength, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

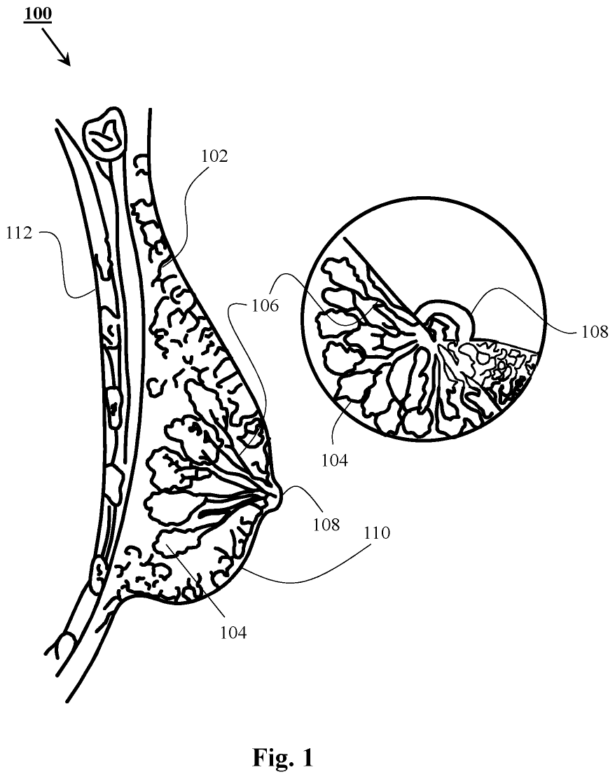

In human beings, mammary glands are located on the anterior chest wall. In human adults, a female mammary gland is more developed than a male mammary gland, as the primary function of a female adult mammary gland is to produce milk for nursing a new-born child. The adult human female mammary gland comprises a plurality of glands that develop during puberty and maturation. Hormones such as estrogen and progesterone promote the growth and changes in the adult human female mammary gland particularly during pregnancy and the menstrual cycle. Referring now to Fig.1, the anatomy of the mammary gland 100 of an adult human female is illustrated. The mammary gland 100 is a large mass of glandular, fatty, and fibrous tissues 102. These glandular tissues 102 are attached to the chest wall by fibrous strands. Further, a layer of fatty tissue surrounds the mammary gland 100 and extends throughout the mammary gland 100. These fatty tissues give the mammary gland 100 a soft consistency.

Further, a well-developed mammary gland 100 of an adult human female comprises a plurality of milk glands 104. These milk glands 104 are also known as lobules in medical terminology. These milk glands 104 are housed in the glandular tissues 102. There is a plurality of ducts 106 in the mammary gland 100 wherein each such duct 106 facilitates the transportation of milk from each of the plurality of milk glands 104. These ducts 106 are housed in the glandular tissues 102. Nearing the nipple 108, each such duct 106 broadens to form a sac-like structure which is known as ampulla in medical terminology. There is an areola 110 which is a pink or brown colored area surrounding the nipple 108. The mammary gland 100 of the adult human female is located on pectoral muscle 112 of the chest wall. The anatomy of the mammary glands 100 has been elucidated to enable a person skilled in the art to completely appreciate the construction and functioning of a wearable device that has been elucidated in the following discussion.

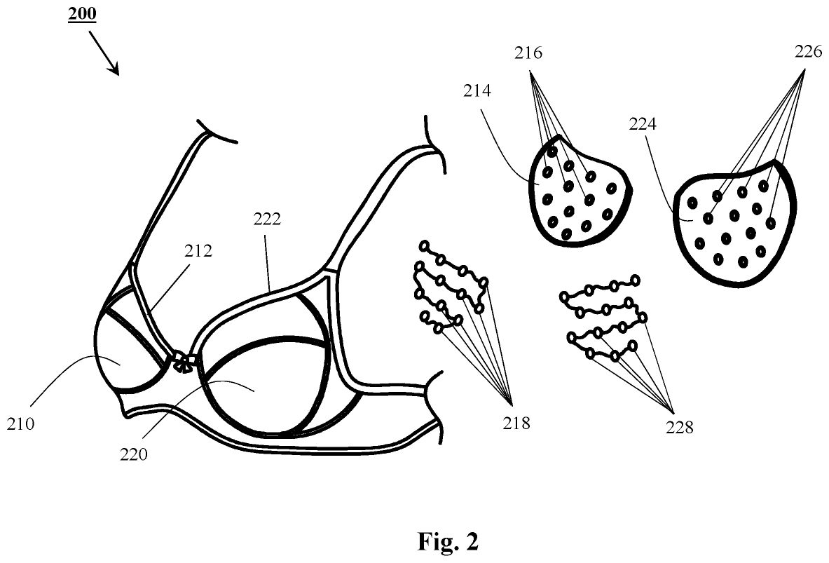

FIG. 2 illustrates an exploded view of a wearable device 200 for mammary glands, in accordance with an embodiment of the present invention. The wearable device 200 includes a first brassiere cup 210 and a second brassiere cup 220. Since the wearable device 200 is envisaged to be directly worn over the mammary glands 100, it is desirable that the materials used for the wearable device 200 be skin-friendly. The first 210 and the second 220 brassiere cups may be made of cotton or nylon or any other suitable material i.e. washable and durable. The first 210 and second 220 brassiere cups are adapted to receive the mammary glands. The first brassiere cup 210 includes a first inner surface 212 and the second brassiere cup 220 includes a second inner surface 222.

The wearable device 200 is also envisaged to include sources of electromagnetic radiation that would be used for therapy and treatment of the mammary glands 100, therefore it is desired that the wearable device 200 be at least partially transparent to the electromagnetic radiations within the range of UV and IR wavelengths, with the UV and IR frequencies included as discussed above. Therefore, a first layer 214 of a diaphanous material is attached to the first inner surface 212 and a second layer 224 of the diaphanous material is attached to the second inner surface 222. The first 214 and the second 224 layers of the diaphanous material are adapted to be in contact with the mammary glands 100. The first 214 and the second 224 layers may be made from transparent silicone materials such as polysiloxane and polyacrylonitrile. A plurality of first openings 216 has been provided in the first layer 214 and a plurality of second openings 226 has been provided in the second layer 214.

A plurality of first Light Emitting Diodes (LEDs) 218 have provided between the first inner surface 212 and the first layer 214, in a manner that the plurality of first LEDs 218 has been provided within the plurality of respective first openings 216. A plurality of second Light Emitting Diodes (LEDs) 228 have provided between the second inner surface 222 and the second layer 224, in a manner that the plurality of second LEDs 228 have been provided within the plurality of respective second openings 226. The LEDs are characterized by their superior power efficiencies, smaller sizes, rapidity in switching, physical robustness, and longevity when compared with incandescent or fluorescent lamps. In that regard, the plurality of first 218 and the second 228 LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high power LEDs, etc. In that manner, the plurality of the first 216 and the second 226 openings facilitate passage of chromatic and non-chromatic light generated by the plurality of the first 218 and the second 228 LEDs, respectively, to allow the chromatic and non-chromatic light to be incident upon the mammary glands 100.

Materials used in the plurality of first 218 and the second 228 LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultra Violet (UV) LEDs may be combined with europium based phosphors to generate red and blue lights and copper and zinc doped zinc sulfide-based phosphor to generate green light.

In addition to conventional mineral-based LEDs, the plurality of first 218 and the second 228 LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N. (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557B2, titled “Roll-to-roll fabricated light sheet and encapsulated semiconductor circuit devices”, which is included herein in its entirety, by reference. In several embodiments, the plurality of first 218 and the second 228 LEDs may also be micro-LEDs described through U.S. Pat. Nos. 8,809,126B2, 8,846,457B2, 8,852,467B2, 8,415,879B2, 8,877,101B2, 9,018,833B2, and their respective family members, assigned to NthDegree Technologies Worldwide Inc., which are included herein by reference, in their entirety. The plurality of first 218 and the second 228 LEDs, in that regard, may be provided as a printable composition of the micro-LEDs, printed on a substrate.

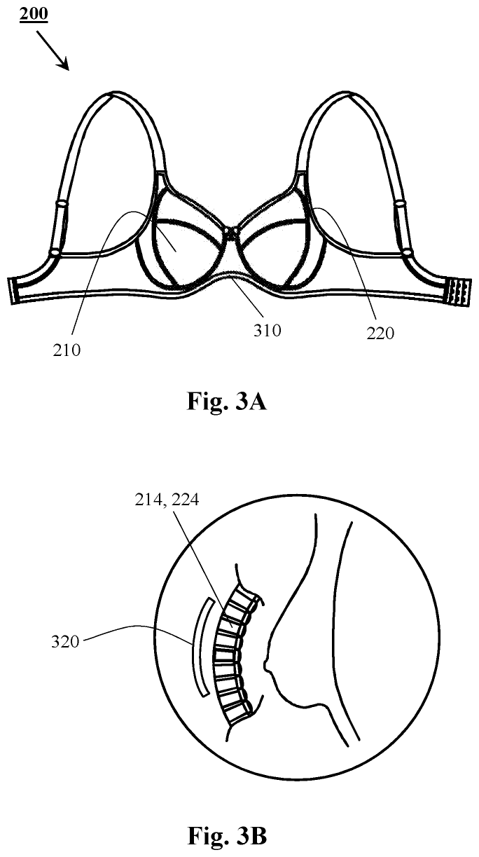

FIG. 3A illustrates a front view of the wearable device 200 of FIG. 2. As illustrated in FIG. 3A, an underwire 310 is provided at the base of the first 210 and the second 220 brassiere cups. The underwire 310, preferably in the semi-circle configuration, provides support and structure for the wearable device 200. FIG. 3B illustrates a partial side sectional view of the wearable device 200 of FIG. 2. A sensor unit 320 has been provided between each of the first inner surface 212 and the first layer 214, and the second inner surface 222 and the second layer 224. The sensor unit 320 is adapted to determine one or more physiological parameters of the user wearing the wearable device 200. The sensor unit 320 may include one or more of a temperature sensor, a proximity sensor, an accelerometer, an infra-red sensor including a passive infrared sensor, a pressure sensor, an ultrasonic sensor, a touch sensor, a color sensor, and a tilt sensor, to name a few. The physiological parameters may include among others, the pulse rate, the blood pressure level, the levels of blood sugar and urine sugar, and the breathing rate.

The diagnosis of the diseases and disorders related to the mammary glands 100 include among others, mammary gland pain, lumps, discharge from the nipple, changes in the mammary gland's skin with the mammary gland skin becoming pitted, puckered, red, thickened, or dimpled. The changes in the mammary glands 100 further include a lump that feels distinctly different from other mammary gland tissue, a lump stuck to the skin or chest wall, a swelling, scaly skin around the nipple, changes in the shape of the mammary gland, nipple fissures during lactation in the postpartum period including the circular nipple fissure and longitudinal nipple fissure wherein a circular nipple fissure is commonly located at the nipple-areolar junction whereas a longitudinal fissure is situated throughout the entire length of the nipple either vertically or horizontally, inflammation of the mammary glands 100 during lactation in the postpartum period, changes in the nipple, and bloody and/or spontaneous discharge from the nipple.

The diagnosis of the diseases and disorders is generally dependent on the analysis of several symptoms, and physiological characteristics of the user, such as the height, the weight, the blood group, and the age, etc. Other factors that may be important include a history of the conditions experienced by the user, family history of disorders, past procedures received, and other factors such as geographical location and presence of environmental contaminants in the direct environment of the user. Therefore, analysis of a large amount of historical data along with data collected by the sensor unit 320 including several aforementioned sensors and data collected through other sources may be utilized within the wearable device 200. It is therefore desirable that a control architecture is provided within the wearable device 200, which would able to automatically determine the nature of the condition that the user is suffering from and appropriate treatment characteristics such as intensity, wavelengths, mode of operation (pulsed or continuous), and duration of the irradiation therapy.

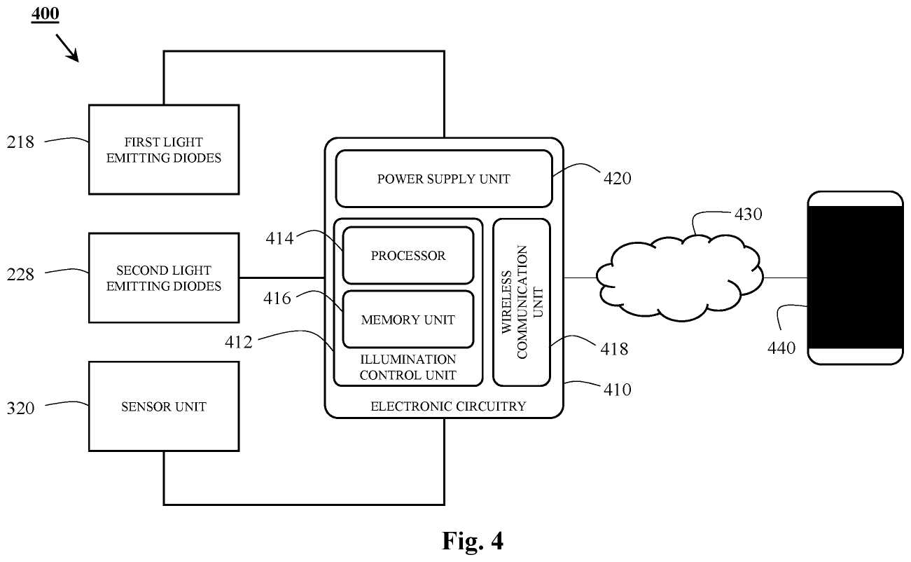

FIG. 4 illustrates a control architecture 400 for monitoring and control of the wearable device 200 of Fig.2, in accordance with an embodiment of the present invention. The control architecture 400 of the wearable device 200 includes an electronic circuitry 410. The electronic circuitry 410 further includes an illumination control unit 412 including a processor 414 and a memory unit 416. The processor 414 may be a general-purpose processor, a Field Programmable Gate Array (FPGA), an Application Specific Integrated Circuit (ASIC), or the like. The memory unit 416 may be EPROM, EEPROM, flash memory, or the like. Further, the electronic circuitry 410 includes a wireless communication unit 418 that may be connected with a network 430 through protocols such as 802.11 (Wireless Fidelity), Near Field Communication (NFC), Bluetooth and ZigBee, etc. Also, the wireless communication unit 418 may further be able to communicate with an external electronic communication device 440, through network 430. The external electronic communication device 440 may include a mobile handheld device such as a smartphone, a tablet, a notebook, or a desktop computer, etc. The electronic circuitry 410 also includes a power supply unit 420 which may be replaceable batteries or rechargeable batteries configured to be charged from wired or wireless means. Additional switches for manual operation or choosing between manual and automatic operations of the wearable device 200 may also be provided in connection with the electronic circuitry 410. Also, all elements of the electronic circuitry 410 may be made from water-resistant materials.

Further connected to the electronic circuitry 410 are the plurality of the first 218 and the second 228 LEDs and the sensor unit 320. The sensor unit 320 is adapted to detect any disorder and diseases pertinent to the mammary glands 100 including but not limited to mammary gland inflammation, lumps, discharge from the nipple, changes in the skin of the mammary glands 100 with the mammary gland skin becoming pitted, puckered, red, thickened, or dimpled. The identification and detection of the variation in the condition of the mammary glands 100, if any, is communicated by the sensor unit 320 to the illumination control unit 412 in the form of a set of electronic communication signals. The illumination control unit 412, is configured to process the set of electronic communication signals received from the sensor unit 320. After processing the signals received from the sensor unit 320, the illumination control unit 412 initiates the operation of the plurality of the first 218 and the second 228 LEDs with required intensity, amplitude, wavelength, and frequency for providing a therapeutic effect to the exposed area of the mammary glands. The illumination control unit 412 is also configured to regulate and control the duration of exposure of the mammary glands to the light emitted from the plurality of the first 218 and the second 228 LEDs.

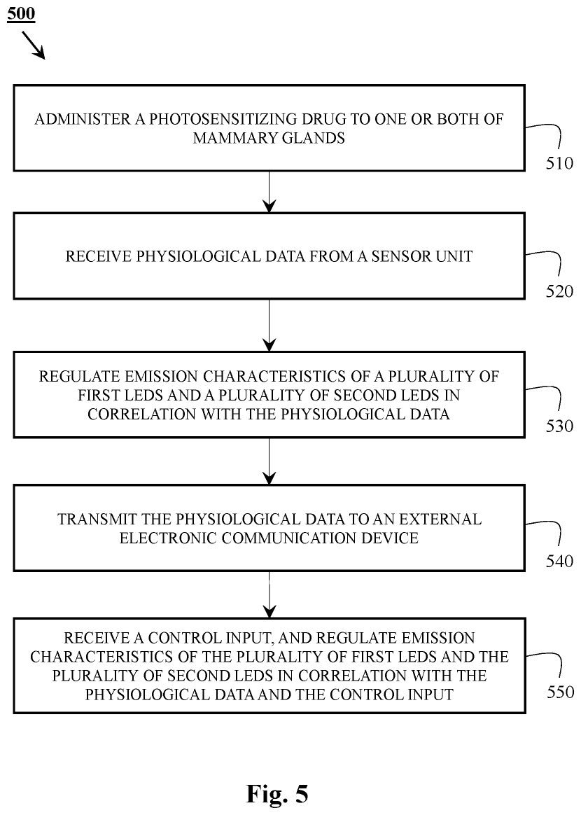

FIG. 5 illustrates a method 500 for utilizing the wearable device 200 of FIG. 2, in accordance with an embodiment of the present invention. The method begins at step 510 when a photosensitizing drug is administered to one or both of the mammary glands of a patient. The photosensitizing drug may include a photosensitizing agent and may be administered through an injection. It is to be noted here that the method 500 is only one of the several possible methods in which the wearable device 200 may be utilized. Therefore, there may be several other methods where the administration of the photosensitizing drug may not be necessary for availing the therapeutic benefits of the wearable device 200.

At step 520, machine-readable instructions stored in the memory unit 416, when executed by the processor 414 causes the processor 414 to receive the physiological data from the sensor unit 320. The physiological data may include one or more of a core body temperature, a pulse rate, a blood pressure level, levels of blood sugar and urine sugar, and breathing rate, of the user.

At step 530, the processor 414 regulates the emission characteristics, such as wavelength, frequency, pulse-width and time period, etc. of the plurality of first LEDs 218 and the plurality of second LEDs 228 in correlation with the physiological data. The photosensitizing agent of the photosensitizing drug releases molecular oxygen when activated by the radiation from the plurality of the first 218 and the second 228 LEDs. This molecular oxygen facilitates the killing of the tumor cells in the affected area of the mammary glands. The usual medical practice is to inject the photosensitizing drug into the bloodstream of the patient. The photosensitizing drug is absorbed by both normal healthy cells and tumor cells in the body of the patient. However, the administered photosensitizing drug stays for a longer duration in tumor cells of the affected area than in normal healthy cells. The photosensitizing drug leaves the healthy normal cells after a time duration of anywhere between 24 hours to 72 hours whereas the photosensitive drug remains for more than 72 hours in tumor cells. The photosensitizing drug in the tumor cells absorb electromagnetic radiation and produces an active form of molecular oxygen that causes the death of nearby tumor cells.

At step 540, the processor 414 transmits the physiological data to the external electronic communication device 440, via the wireless communication unit 418 and the network 430. At step 550, the processor 414 receives a control input from the external electronic communication device 440 and regulates the emission characteristics of the plurality of first LEDs 218 and the plurality of second LEDs 228 in correlation with the physiological data and the control input. In this manner, a user, such as a physician or a patient would be able to control the treatment parameters for the treatment of any specific condition in the mammary glands.

The present invention provides effective light-based therapy for the treatment of mammary gland related disorders and diseases. The therapy as disclosed in the present invention is a non-invasive, pain-free therapy with little or no side effects. Further, the therapy is simple to use and provides preventive care of the mammary glands against a plurality of pertinent conditions, disorders and diseases of the mammary gland including but not limited to mammary gland pain, lumps, discharge from the nipple, changes in the mammary gland's skin with the mammary gland skin becoming pitted, puckered, red, thickened, or dimpled, a lump that feels distinctly different from other mammary gland tissue, a lump stuck to the skin or chest wall, swelling, scaly skin around the nipple, changes in the shape of the mammary gland, nipple fissures during lactation in the postpartum period, inflammation of the mammary glands during lactation in the postpartum period, changes in the nipple and discharge from the nipple, especially if it is bloody and/or occurs spontaneously.

The programming instructions can be, for example, computer-executable and/or logic implemented instructions. In some examples, a computing device is configured to provide various operations, functions, or actions in response to the programming instructions conveyed to the computing device by one or more of the computer-readable medium, the computer recordable medium, and/or the communications medium. The non-transitory computer-readable medium can also be distributed among multiple data storage elements, which could be remotely located from each other. The computing device that executes some or all of the stored instructions can be a micro-fabrication controller or another computing platform. Alternatively, the computing device that executes some or all of the stored instructions could be remotely located computer systems, such as a server.

Further, while one or more operations have been described as being performed by or otherwise related to certain modules, devices or entities, the operations may be performed by or otherwise related to any module, device or entity. As such, any function or operation that has been described as being performed by a module could alternatively be performed by a different server, by the cloud computing platform, or a combination thereof. Further, the operations need not be performed in the disclosed order, although in some examples, an order may be preferred. Also, not all functions need to be performed to achieve the desired advantages of the disclosed system and method, and therefore not all functions are required.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

1. A wearable device for mammary glands in human beings, the wearable device comprising:
a first brassiere cup and a second brassiere cup, the first brassiere cup including a first inner surface and the second brassiere cup including a second inner surface;
a first layer of a diaphanous material attached to the first inner surface and a second layer of the diaphanous material attached to the second inner surface, wherein the first and the second layers are adapted to be in contact with the mammary glands;
a plurality of first openings provided in the first layer and a plurality of second opening provided in the second layer;
a plurality of first Light Emitting Diodes (LEDs) provided between the first inner surface and the first layer, the plurality of first LEDs provided within the plurality of respective first openings;
a plurality of second LEDs provided between the second inner surface and the second layer, the plurality of second LEDs provided within the plurality of respective second openings;
a sensor unit adapted to determine one or more physiological parameters; and
an underwire connecting the first and the second brassiere cups.

2. The wearable device as claimed in claim 1, wherein the plurality of first LEDs and the plurality of second LEDs are configured to emit the electromagnetic radiation in one or more of a pulse mode and continuous mode.

3. The wearable device as claimed in claim 1, wherein the plurality of first LEDs and the plurality of second LEDs have been provided on an Organic LED (OLED) based flexible panel or an inorganic LED based flexible panel.

4. The wearable device as claimed in claim 1, wherein the plurality of first LEDs and the plurality of second LEDs are provided as a printable composition of micro-LEDs, printed on a substrate.

5. The wearable device as claimed in claim 1, further comprising a processor, a memory unit and a communication unit, the memory unit including machine-readable instructions that when executed by the processor, enable the processor to:
receive physiological data from the sensor unit; and
regulate emission characteristics of the plurality of first LEDs and the plurality of second LEDs in correlation with the physiological data.

6. The wearable device as claimed in claim 5, wherein the processor is further enabled to transmit the physiological data to an external electronic communication device, via the communication unit.

7. The wearable device as claimed in claim 5, wherein the processor is further enabled to receive a control input, and regulate emission characteristics of the plurality of first LEDs and the plurality of second LEDs in correlation with the physiological data and the control input.

8. A method of utilizing a wearable device for mammary glands in human beings, the wearable device comprising:
a first brassiere cup and a second brassiere cup, the first brassiere cup including a first inner surface and the second brassiere cup including a second inner surface,
a first layer of a diaphanous material attached to the first inner surface and a second layer of the diaphanous material attached to the second inner surface, wherein the first and the second layers are adapted to be in contact with the mammary glands,
a plurality of first openings provided in the first layer and a plurality of second opening provided in the second layer,
a plurality of first Light Emitting Diodes (LEDs) provided between the first inner surface and the first layer, the plurality of first LEDs provided within the plurality of respective first openings,
a plurality of second LEDs provided between the second inner surface and the second layer, the plurality of second LEDs provided within the plurality of respective second openings,
a sensor unit adapted to determine one or more physiological parameters, and an underwire connecting the first and the second brassiere cups;
the method comprising steps of:
administering a photosensitizing drug to one or both of the mammary glands;
receiving physiological data from the sensor unit; and
regulating emission characteristics of the plurality of first LEDs and the plurality of second LEDs in correlation with the physiological data.

9. The method as claimed in claim 8, further comprising a step of transmitting the physiological data to an external electronic communication device.

10. The method as claimed in claim 8, further comprising a step of receiving a control input, and regulating emission characteristics of the plurality of first LEDs and the plurality of second LEDs in correlation with the physiological data and the control input.
